(19) United States
(12) Patent Application Publication
(10) Pub. No.: US 2007/0270356 A1
(43) Pub. Date: Nov. 22, 2007

(54) Substituted 9A-N-[N'-(Benzenesulfonyl)Carbamoyl-Y-Aminopropyl] and 9A-N-[N'(B-Cyanoethyl)-N'-(Benzenesulfonyl)Carbamoyl-Y-Aminopropyl]Derivatives of 9-Deoxo-9-Dihydro-9A-Aza-9A-Homoerithomycin A

(75) Inventors: Nedjeljko Kujundzic, Zagreb (HR); Mirjana Bukvic Krajacic, Zagreb (HR); Karmen Brajsa, Zagreb (HR)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXOSMITHKLINE ISTRAZIVACKI CENTAR ZAGREB D.O.O., Zagreb (HR)

(21) Appl. No.: 10/534,261

(22) PCT Filed: Nov. 10, 2003
(86) PCT No.: PCT/HR03/00057

§ 371 Date: Nov. 30, 2005

(30) Foreign Application Priority Data
Nov. 11, 2002 (HR) P20020886A

(51) Int. Cl. A61K 31/7048; A61P 31/04; C07H 17/08

(52) U.S. Cl. 514/25; 536/7.1

(57) ABSTRACT

The invention relates to substituted 9a-N—[N′-(benzenesulfonyl)carbamoyl-γ-aminopropyl] and 9a-N—[N′-(β-cyanoethyl)-N′-(benzenesulfonyl)carbamoyl-γ-aminopropyl] derivatives of 9-deoxo-9-dihydro-9a-aza-9a-homoerithromycin A and 5-( )-desosaminyl-9-deoxo-9-dihydro-9a-aza-9a-homoerithronolide A, novel semisynthetic macrolide antibiotics of the azalide series, of the formula (I) wherein R represents H or cladinosyl moiety, R¹ represents H or β-cyanoethyl group an R² represents II or fluoro, chloro and methyl group, and pharmaceutically acceptable salts thereof with inorganic or organic acids, to the process for the preparation of pharmaceutical compositions as well as to the use their compositions for sterilization rooms and medical instruments as well as for protection of wall and wooden coatings.

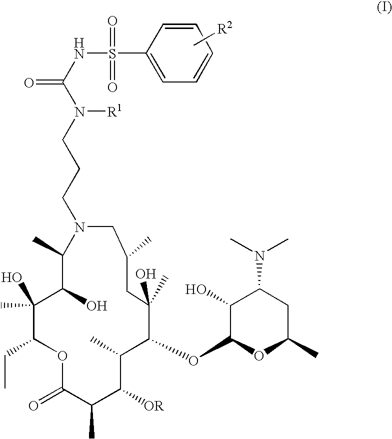

### TECHNICAL FIELD

Int. Cl. C 07H 17/08, A61K 31/71

### TECHNICAL PROBLEM

The present invention relates to substituted 9a-N—[N′-(benzenesulfonyl)carbamoyl-γ-aminopropyl] and 9a-N—[N′-(β-cyanoethyl)-N′-(benzenesulfonyl)carbamoyl-γ-aminopropyl] derivatives of 9-deoxo-9-dihydro-9a-aza-9a-homoerithromycin A and 5-O-desosaminyl-9-deoxo-9-dihydro-9a-aza-9a-homoerithronolide A, novel semisynthetic macrolide antibiotics of the azalide series having antibacterial activity, general formula 1, 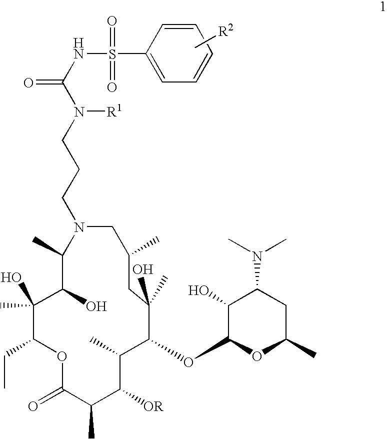
wherein R represents H or cladinosyl moiety, R¹ represents H or β-cyanoethyl group and R² represents H or fluoro, chloro and methyl group, to pharmaceutically acceptable addition salts thereof with inorganic or organic acids, to a process for the preparation of the pharmaceutical compositions as well as to the use of these compositions for the sterilization the rooms and the medicinal instruments, as well as for the prevention of walls and wooden materials.

### PRIOR ART

Erithromycin A is a macrolide antibiotic, whose structure is characterized by 14-membered macrolactone ring having carbonyl group in C-9 position. It was found by McGuire in 1952 [Anitibiot. Chemother., 2 (1952) 281] and for over 40 years it has been considered as a reliable and effective antimicrobial agent in the treatment of diseases caused by Gram-positive and some Gram-negative microorganisms. However, in an acidic medium it is easily converted into anhydroerythromycin A, an inactiv C-6/C-12 metabolite of a spiroketal structure [P. Kurath et al., Experientia 27 (1971) 362].It is well-known that spirocyclisation of aglycone ring of erythromycin A is successfully inhibited by a chemical transformation of C-9 ketones or hydroxy groups in C-6 and/or C-12 position. By the oximation of C-9 ketones [S. Dokić et al., Tetrahedron Lett. 1967: 1945] and by subsequently modifying the obtained 9(E)-oxime into 9-[O-(2-methoxyethoxy)methyloxime]erithromycin A (ROXITHROMYCIN) [G. S. Ambrieres, Fr. pat. 2,473,525, 1981] or 9(S)-erithromycylamine [R. S. Egan et al., J. Org. Chem. 39 (1974) 2492] or a more complex oxazine derivative thereof, 9-deoxo-11-deoxy-9,11-{imino[2-(2-methoxyethoxyethylidene]oxy}-9(S)-erythromycin A (DIRITHROMYCIN) [P. Lugar et al., J. Crist. Mol. Strict. 9 (1979) 329], novel semisynthetic macrolides were synthesized, whose basic characteristic, in addition to a greater stability in an acidic medium, is a better pharmacokinetics and a long half-time with regard to the parent antibiotic erythromycin A. In a third way for modifying C-9 ketones use is made of Beckmann rearrangement of 9(E)-oxime and of a reduction of the obtained imino ether (G. Kobrehel et al., U.S. Pat. No. 4,328,334, 1982.) into 11-aza-10-deoxo-10-dihydroerythromycin A (9-deoxo-9a-aza-9a-homoerythromycin A) under broadening the 14-member ketolactone ring into a 15-member azalactone ring. By reductive N-methylation of 9a-amino group according to Eschweiler-Clark process (G. Kobrehel et al., BE Pat. 892,397, 1982.) or by a preliminary protection of amino group by means of conversion into the corresponding N-oxides and then by alkylation and reduction [G. M. Bright, U.S. Pat. No., 4,474,768, 1984.] N-methyl-11-aza-10-deoxo-10-dihydroerythromycin A (9-deoxo-9a-methyl-9a-aza-9a-homoerithromycin A, AZITHROMYCIN) was syntetized, a prototype of azalide antibiotics, which, in addition to a broad antimicrobial spectrum including Gram-negative bacteria and intracellular microorganisms, are characterized by a specific mechanism of transport to the application site, a long biological half-time and a short therapy period. In EP A 0316128 (Bright G. M. et al.) novel 9a-allyl and 9a-propargyl derivatives of 9-deoxo-9a-aza-9a-homoerythromycin A are disclosed and in U.S. Pat. No. 4,492,688, from 1985 (Bright G. M.) the synthesis and the antibactertial activity of the corresponding cyclic ethers are disclosed. In the there are further disclosed the synthesis and the activity spectrum of novel 9-deoxo-9a-aza-11-deoxy-9a-homoerythromycin A 9a,11-cyclic carbamates and O-methyl derivatives thereof (G. Kobrehel et al., J. Anitibiot. 46 (1993) 1239-1245).

By reaction of 9-deoxo-9-dihydro-9a-aza-9a-homoerithromycin A with isocyanates or isothiocyanates respectively [N. Kujund\check{z}ić et al. Croat. Pat. 931480, 1993.], 9a-N-(N′-carbamoyl) and 9a-N-(N′-thiocarbamoyl) derivatives of 9-deoxo-9-dihydro-9a-aza-9a-homoerithromycin A with a certain antibacterial activity are obtained.

According to the known and established Prior Art, 9a-N—[N′-(benzenesulfonyl)carbamoyl-γ-aminopropyl] and 9a-N—[N′-(β-cyanoethyl)-N′-(benzenesulfonyl)carbamoyl-γ-aminopropyl] derivatives of 9-deoxo-9-dihydro-9a-aza-9a-homoerithromycin A and 5-O-desosaminyl-9-deoxo-9-dihydro-9a-aza-9a-homoerithronolide A and pharmaceutically acceptable addition salts thereof with inorganic or organic acids, a process for the preparation thereof as well as the preparation methods and use an pharmaceutical preparations have not been disclosed as yet.

It has been found and it is object of the present invention, that 9a-N—[N′-(benzene-sulfonyl)carbamoyl-γ-aminopropyl] and 9a-N—[N′-(β-cyanoethyl)-N′-(benzenesulfonyl)-carbamoyl-γ-aminopropyl] derivatives of 9-deoxo-9-dihydro-9a-aza-9a-homoerithromycin A and 5-O-desosaminyl-9-deoxo-9-dihydro-9a-aza-9a-homoerithronolide A, novel semisynthetic macrolide antibiotics of the azalide series and pharmaceutically acceptable addition salts thereof with inorganic or organic acids may be prepared by reacting 9a-N-(γ-aminopropyl) or 9a-N—[N′-(β-cyanoethyl)-γ-aminopropyl] derivatives of 9-deoxo-9-dihydro-9a-aza-9a-homoerithromycin A and 5-O-desosaminyl-9-deoxo-9-dihydro-9a-aza-9a-homoerithronolide A with phenylsulfonylisocyanate and optionally by reacting the obtained 9a-N—[N′-(benzenesulfonyl)carbamoyl-γ-aminopropyl] and 9a-N—[N′-(β-cyanoethyl)-γ-aminopropyl]-derivatives of 9-deoxo-9-dihydro-9a-aza-9a-homoerithromycin A and 5-O-desosaminyl-9-deoxo-9-dihydro-9a-aza-9a-homoerithronolide A with inorganic and organic acids.

Technical Solution

It has been found that novel 9a-N—[N′-(benzenesulfonyl)carbamoyl-(N′-benzenesulfonyl)carbamoyl-γ-aminopropyl] and 9a-N—[N′-(β-cyanoethyl)-N′-(benzenesulfonyl)carbamoyl-γ-aminopropyl] derivatives of 9-deoxo-9-dihydro-9a-aza-9a-homoerithromycin A and 5-O-desosaminyl-9-deoxo-9-dihydro-9a-aza-9a-homoerithronolide A of the general formula 1, wherein R represents H or cladinosyl group, R¹ represents H or β-cyanoethyl moiety and R² represents H or fluoro, chloro and methyl group 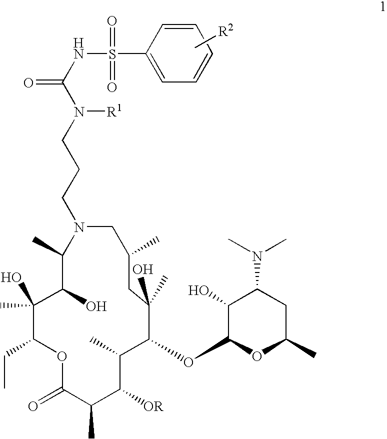
may be prepared by reacting 9a-N-(γ-aminopropyl) and 9a-N—[N′-(β-cyanoethyl)-γ-aminopropyl] derivatives of 9-deoxo-9-dihydro-9a-aza-9a-homoerithromycin A and 5-O-desosaminyl-9-deoxo-9-dihydro-9a-aza-9a-homoerithronolide A general formula 2, 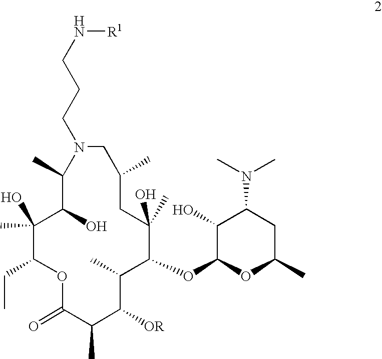
wherein R represents H or cladinosyl group and R¹ represents H or β-cyanoethyl moiety, with the substituted phenylsulfonylisocyanates general formula 3, 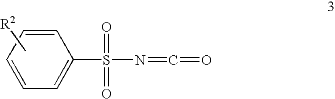
wherein R² represents H or fluoro, chloro and methyl group, in toluene, xylene or some other aprotic solvent, at a temperature 0° to 110° C.

Pharmaceutically acceptable acid addition salts, which also represents an object of the present invention are obtained by reacting 9a-N—[N′-(benzenesulfonyl)carbamoyl-γ-aminopropyl] and 9a-N—[N′-(β-cyanoethyl)-N′-(benzenesulfonyl)carbamoyl-γ-aminopropyl] derivatives of 9-deoxo-9-dihydro-9a-aza-9a-homoerithromycin A and 5-O-desosaminyl-9-deoxo-9-dihydro-9a-aza-9a-homoerithronolide A with an at least equimolar amount of the corresponding inorganic or organic acid such as hydrochloric acid, hydroiodic acid, sulfuric acid, phosphoric acid, acetic acid, trifluoroacetic acid, propionic acid, benzoic acid, benzene sulfonic acid, methane sulfonic acid, lauryl sulfonic acid, stearic acid, palmitic acid, succinic acid, ethylsuccinic acid, lactobionic acid, oxalic acid, salicylic acid and similar acid, in a solvent inert to the reaction. Addition salts are isolated by evaporating the solvent or, alternatively, by filtration after a spontaneous precipitation or a precipitation by the addition of a non-polar cosolvent.

9a-N—[N′-(Benzenesulfonyl)carbamoyl-γ-aminopropyl] and 9a-N—[N′-(β-cyanoethyl)-N′-(benzenesulfonyl)carbamoyl-γ-aminopropyl] derivatives of 9-deoxo-9-dihydro-9a-aza-9a-homoerithromycin A and 5-O-desosaminyl-9-deoxo-9-dihydro-9a-aza-9a-homoerithronolide A of the general formula 1 and pharmaceutically acceptable addition salts with inorganic or organic acids thereof possess an antibacterial activity in vitro. Minimal inhibitory concentration (MIC) is defined as the concentration which shows 90% growth inhibition, and was determinated by broth dilution methods National Committee for Clinical Laboratory Standards (NCCLS, M7-A2 protocols). Final concentration of test substances were in range from 64 to 0.125 mg/l. MIC levels for all compound were determinated on panel of susceptible and resistant Gram positive bacterial strains (S. aureus, S. pneumoniae and S. pyogenes) and on Gram negative strains (E. coli, H. influenzae, E. faecalis, M. catarrhalis).

Test substances from Examples 1 to 7 and 15 to 21 were active on susceptible strains of S. pyogenes (MIC 0.125 to 4.0 mg/l), and on susceptible strains on S. pneumoniae (MIC 0.125 to 8.0 mg/l). MIC values on susceptible S. aureus strains were from 1 to 16 mg/l. Substances from Examples 1 to 7 and 15 to 21 showed strong antimicrobial activities on most tested Gram negative strains; M. catarrhalis MIC from 0.25 to 16 mg/l, E. coli from 8 to 16 mg/l, E. faecalis from 2 to 8 mg/l.

The obtained results for substances from Example 1 to 7 and 15 to 21 expressed as MIC in mg/l suggest a potential use thereof as sterilization agents of e.g. rooms and medical instruments and as industrial microbial agents e.g. for the protection of wall and wooden coatings.

Process for the preparation of 9a-N—[N′-(benzenesulfonylcarbamoyl)-γ-aminopropyl] and 9a-N—[N′-(β-cyanoethyl)-N′-(benzenesulfonyl)carbamoyl-γ-aminopropyl] derivatives of 9-deoxo-9-dihydro-9a-aza-9a-homoerithromycin A and 5-O-desosaminyl-9-deoxo-9-dihydro-9a-aza-9a-homoerithronolide A of this invention is illustrated by the following Examples which should in no way be construed as a limitation of the scope thereof.

### EXAMPLE 1

### 9-Deoxo-9-dihydro-9a-N—[N′-(p-toluensulfonyl)carbamoyl-γ-aminopropyl]-9a-aza-9a-homoerithromycin A

A mixture of 1.0 g (1.26 mmol) 9-deoxo-9-dihydro-9a-aza-9a-(γ-aminopropyl)-9a-homoerithromycin A and 0.26 g (1.3 mmol) of p-toluensulfonylisocyanate in 30 ml dry toluene was stirred for on 1.0 hour at the temperature 0-5° C. to complete the reaction. The crystals of the crude product were filtered, wherefrom by chromatography on silica gel column using the solvent system methylene-chloride:methanol=7:3, pure 9-deoxo-9-dihydro-9a-N—[N′-(p-toluensulfonyl)carbamoyl-γ-aminopropyl]-9a-aza-9a-homoerithromycin A was obtained.

MS(ES⁺)m/z=989.

### EXAMPLE 2

### 9-Deoxo-9-dihydro-9a-N—[N′-(4-chlorobenzenesulfonyl)carbamoyl-γ-aminopropyl]-9a-aza-9a-homoerithromycin A

From 1.0 g (1.26 mmol) 9-deoxo-9-dihydro-9a-aza-9a-(γ-aminopropyl)-9a-homoerithromycin A and 0.28 g (1.3 mmol) of 4-chlorobenzenesulfonylisocyanate in 20 ml dry toluene the crude product was obtained, wherefrom by chromatography on silica gel column using the solvent system methylene-chloride:methanol=1:1, pure 9-deoxo-9-dihydro-9a-N—[N′-(4-chloro-benzenesulfonyl)carbamoyl-γ-aminopropyl]-9a-aza-9a-homoerithromycin A was obtained.

MS(ES⁺)m/z=1009.

### EXAMPLE 3

### 9-Deoxo-9-dihydro-9a-N—[N′-(benzenesulfonyl)carbamoyl-γ-aminopropyl]-9a-aza-9a-homoerithromycin A

From 1.01 g (1.28 mmol) 9-deoxo-9-dihydro-9a-aza-9a-(γ-aminopropyl)-9a-homoerithromycin A and 0.23 g (1.91 mmol) of benzenesulfonylisocyanate in 20 ml dry toluene the crude product was obtained, wherefrom by chromatography on silica gel column using the solvent system methylene-chloride:methanol=1:1, pure 9-deoxo-9-dihydro-9a-N—[N′-(benzenesulfonyl)-carbamoyl-γ-aminopropyl]-9a-aza-9a-homoerithromycin A was obtained.

MS(ES⁺)m/z=975.

### EXAMPLE 4

### 9-Deoxo-9-dihydro-9a-N—[N′-(o-toluenesulfonyl)carbamoyl-γ-aminopropyl]-9a-aza-9a-homoerithromycin A

From 1.0 g (1.26 mmol) 9-deoxo-9-dihydro-9a-aza-9a-(γ-aminopropyl)-9a-homoerithromycin A and 0.26 g (1.3 mmol) of o-toluensulfonylisocyanate in 20 ml dry toluene the crude product was obtained, wherefrom by chromatography on silica gel column using the solvent system methylene-chloride:methanol=1:1, pure 9-deoxo-9-dihydro-9a-N—[N′-(o-toluensulfonyl)-carbamoyl-γ-aminopropyl]-9a-aza-9a-homoerithromycin A was obtained.

MS(ES⁺)m/z=989.

### EXAMPLE 5

### 9-Deoxo-9-dihydro-9a-N—[N′-(2-chlorobenzenesulfonyl)carbamoyl-γ-aminopropyl]-9a-aza-9a-homoerithromycin A

From 1.0 g (1.26 mmol) 9-deoxo-9-dihydro-9a-aza-9a-N-(γ-aminopropyl)-9a-homoerithromycin A and 0.28 g (1.3 mmol) of 2-chlorobenzenesulfonylisocyanate in 20 ml dry toluene the crude product was obtained, wherefrom by chromatography on silica gel column using the solvent system methylene-chloride:methanol=7:3, pure 9-deoxo-9-dihydro-9a-N—[N′-(2-chlorobenzenesulfonyl)carbamoyl-γ-aminopropyl]-9a-aza-9a-homoerithromycin A was obtained.

MS(ES⁺)m/z=1009.

### EXAMPLE 6

### 9-Deoxo-9-dihydro-9a-N—[N′-(4-fluorobenzenesulfonyl)carbamoyl-γ-aminopropyl]-9a-aza-9a-homoerithromycin A

From 1.0 g (1.26 mmol) 9-deoxo-9-dihydro-9a-N-(γ-aminopropyl)-9a-aza-9a-homoerithromycin A and 0.28 g (1.3 mmol) of 4-fluorobenzenesulfonylisocyanate in 20 ml dry toluene the crude product was obtained, wherefrom by chromatography on silica gel column using the solvent system methylene-chloride:methanol=7:3, pure 9-deoxo-9-dihydro-9a-N—[N′-(4-fluorobenzenesulfonyl)carbamoyl-γ-aminopropyl]-9a-aza-9a-homoerithromycin A was obtained.

MS(ES⁺)m/z=993.

### EXAMPLE 7

### 5-O-Desozaminyl-9-deoxo-9-dihydro-9a-N-(γ-aminopropyl)-9a-aza-9a-homoerithronolide A

The suspension of 10. 0 g (12.6 mmol) 9-deoxo-9-dihydro-9a-N-(γ-aminopropyl)-9a-aza-9a- homoerithromycin A 120 ml of hydrochloric acid (10%) was stirred for 24 hours at a room temperature, the pH was adjusted to 9.5-10 by adding 5 N sodium hydroxide solution and was extracted with methylene chloride (3×40 ml). The combined organic layers was washed with water (2×50 ml), dried over anhydrous sodium sulfate, evaporated to dryness under reduced pressure to give crude product wherefrom by chromatography on silica gel column using the solvent system methylene chloride:methanol=7:3, pure 5-O-desozaminyl-9-deoxo-9-dihydro-9a-N-(γ-aminopropyl)-9a-aza-9a-homoerithronolide A was obtained.

MS(ES⁺)m/z=653.

### EXAMPLE 8

### 5-O-Desozaminyl-9-deoxo-9-dihydro-9a-N—[N′-(p-toluenesulfonyl)carbamoyl-γ-aminopropyl]-9a-aza-9a-homoerithronolide A

From 1.0 g (1.26 mmol) 5-O-desozaminyl-9-deoxo-9-dihydro-9a-N-(γ-aminopropyl)-9a-aza-9a-homoerithronolide A and 0.34 g (1.73 mmol) of p-toluenesulfonylisocyanate in 20 ml dry toluene the crude product was obtained, wherefrom by chromatography on silica gel column using the solvent system methylene-chloride:methanol: 25% ammonia=90:20:1.5, pure 5-O-desozaminyl-9-deoxo-9-dihydro-9a-N—[N′-(p-toluenesulfonyl)carbamoyl-γ-aminopropyl]-9a-aza-9a-homoerithronolide A was obtained.

MS(ES⁺)m/z=831.

### EXAMPLE 9

### 5-O-Desozaminyl-9-deoxo-9-dihydro-9a-N—[N′-(4-chlorobenzenesulfonyl)carbamoyl-γ-aminopropyl]-9a-aza-9a-homoerithronolide A

From 1.0 g (1.57 mmol) 5-O-desozaminyl-9-deoxo-9-dihydro-9a-N-(γ-aminopropyl)-9a-aza-9a-homoerithronolide A and 0.36 g (1.765 mmol) of 4-chlorobenzenesulfonylisocyanate in 20 ml dry toluene the crude product was obtained, wherefrom by chromatography on silica gel column using the solvent system methylene-chloride:methanol: 25% ammonia=90:20:1.5, pure 5-O-desozaminyl-9-deoxo-9-dihydro-9a-N—[N′-(4-chlorobenzenesulfonyl)-carbamoyl-γ-aminopropyl]-9a-aza-9a-homoerithronolide A was obtained.

MS(ES⁺)m/z=851.

### EXAMPLE 10

### 5-O-Desozaminyl-9-deoxo-9-dihydro-9a-N—[N′-(4-fluorobenzenesulfonyl)carbamoyl-γ-aminopropyl]-9a-aza-9a-homoerithronolide A

From 1.0 g (1.57 mmol) 5-O-desozaminyl-9-deoxo-9-dihydro-9a-N-(γ-aminopropyl)-9a-aza-9a-homoerithronolide A and 0.35 g (1.73 mmol) of 4-fluorobenzenesulfonylisocyanate in 25 ml dry toluene the crude product was obtained, wherefrom by chromatography on silica gel column using the solvent system methylene-chloride:methanol: 25% ammonia=90:20:1.5 pure 5-O-desozamninyl-9-deoxo-9-dihydro-9a-N—[N′-(4-fluorobenzenesulfonyl)carbamoyl-γ-aminopropyl]-9a-aza-9a-homoerithronolide A was obtained.

MS(ES⁺)m/z=835.

### EXAMPLE 11

### 5-O-Desozaminyl-9-deoxo-9-dihydro-9a-N—[N′-(benzenesulfonyl)carbamoyl-γ-aminopropyl]-9a-aza-9a-homoerithronolide A

From 1.0 g (1.57 mmol) 5-O-desozaminyl-9-deoxo-9-dihydro-9a-N-(γ-aminopropyl)-9a-aza-9a-homoerithronolide A and 0.30 g (1.65 mmol) of benzenesulfonylisocyanate in 25 ml dry toluene the crude product was obtained, wherefrom by chromatography on silica gel column using the solvent system methylene-chloride:methanol: 25% ammonia=90: 20:1.5, pure 5-O-desozaminyl-9-deoxo-9-dihydro-9a-N—[N′-(benzenesulfonyl)carbamoyl-γ-aminopropyl]-9a-aza-9a-homoerithronolide A was obtained.

MS(ES⁺)m/z=817.

### EXAMPLE 12

### 5-O-Desozaminyl-9-deoxo-9-dihydro-9a-N—[N′-(o-toluenesulfonyl)carbamoyl-γ-aminopropyl]-9a-aza-9a-homoerithronolide A

From 1.0 g (1.57 mmol) 5-O-desozaminyl-9-deoxo-9-dihydro-9a-N-(γ-aminopropyl)-9a-aza-9a-homoerithronolide A and 0.33 g (1.65 mmol) of o-toluenesulfonylisocyanate in 25 ml dry toluene the crude product was obtained, wherefrom by chromatography on silica gel column using the solvent system methylene-chloride:methanol: 25% ammonia=90:20:1.5, pure 5-O-desozaminyl-9-deoxo-9-dihydro-9a-N—[N′-(p-toluenesulfonyl)carbamoyl-γ-aminopropyl]-9a-aza-9a-homoerithronolide A was obtained.

MS(ES⁺)m/z=831.

### EXAMPLE 13

### 5-O-Desozaminyl-9-deoxo-9-dihydro-9a-N—[N′-(2-chlorobenzenesulfonyl)carbamoyl-γ-aminopropyl]-9a-aza-9a-homoerithronolide A

From 1.0 g (1.57 mmol) 5-O-desozaminyl-9-deoxo-9-dihydro-9a-N-(γ-aminopropyl)-9a-aza-9a-homoerithronolide A and 0.33 g (1.65 mmol) of 2-chlorobenzenesulfonylisocyanate in 25 ml dry xylene the crude product was obtained, wherefrom by chromatography on silica gel column using the solvent system methylene-chloride:methanol: 25% ammonia=90:20:1.5, pure 5-O-desozaminyl-9-deoxo-9-dihydro-9a-N—[N′-(2-chlorobenzenesulfonyl)carbamoyl-γ-aminopropyl]-9a-aza-9a-homoerithronolide A was obtained.

MS(ES⁺)m/z=851.

### EXAMPLE 14

### 9-Deoxo-9-dihydro-9a-N—[N′-(β-cyanoethyl)-γ-aminopropyl]-9a-aza-9a-homoerithromycin A

The solution of 10.0 g (15.7 mmol) 9-deoxo-9-dihydro-9a-N-(γ-aminopropyl)-9a-aza-9a-homoerithromycin A and 1.0 ml (18.0 mmol) acrylonitrile in 200 ml methanola was heated at the boiling temperature for a 10 hours and evaporated to dryness and the crude product was obtained where from by chromatography on silica gel column using the solvent system methylene chloride:methanol: 25% ammonia=90:9:1.5 pure 9-deoxo-9-dihydro-9a-N—[N′-(β-cyanoethyl)-γ-aminopropyl]-9a-aza-9a-homoerithromycin A was obtained.

MS(ES⁺)m/z=877.

### EXAMPLE 15

### 9-Deoxo-9-dihydro-9a-N—[N′-(β-cyanoethyl)-N′-(p-toluenesulfonyl)carbamoyl-γ-aminopropyl]-9a-aza-9a-homoerithromycin A

From 1.0 g (1.18 mmol) 9-deoxo-9-dihydro-9a-N—[N′-(β-cyanoethyl)-γ-aminopropyl]-9a-aza-9a-homoerithromycin A and 0.25 g (1.25 mmol) of p-toluenesulfonylisocyanate in 25 ml dry toluene the crude product was obtained, wherefrom by chromatography on silica gel column using the solvent system methylene chloride:methanol: 25% ammonia=90:9:1.5, pure 9-deoxo-9-dihydro-9a-N—[N′-(β-cyanoethyl)-N′-(p-toluenesulfonyl)carbamoyl-γ-aminopropyl]-9a-aza-9a-homoerithromycin A was obtained.

MS(ES⁺)m/z=1042.

### EXAMPLE 16

### 9-Deoxo-9-dihydro-9a-N—[N′-(β-cyanoethyl)-N′-(o-toluenesulfonyl)carbamoyl-γ-aminopropyl]-9a-aza-9a-homoerithromycin A

From 1.0 g (1.18 mmol) 9-deoxo-9-dihydro-9a-N—[N′-(β-cyanoethyl)-γ-aminopropyl]-9a-aza-9a-homoerithromycin A and 0.25 g (1.25 mmol) of o-toluenesulfonylisocyanate in 25 ml dry toluene the crude product was obtained, wherefrom by chromatography on silica gel column using the solvent system methylene chloride:methanol: 25% ammonia=90:9:1.5, pure 9-deoxo-9-dihydro-9a-N—[N′-(β-cyanoethyl)-N′-(o-toluenesulfonyl)carbamoyl-γ-aminopropyl]-9a-aza-9a-homoerithromycin A was obtained.

MS(ES⁺)m/z=1042.

### EXAMPLE 17

### 9-Deoxo-9-dihydro-9a-N—[N′-(β-cyanoethyl)-N′-(4-chlorobenzenesulfonyl)carbamoyl-γ-aminopropyl]-9a-aza-9a-homoerithromycin A

From 1.0 g (1.18 mmol) 9-deoxo-9-dihydro-9a-N—[N′-(β-cyanoethyl)-γ-aminopropyl]-9a-aza-9a-homoerithromycin A and 0.27 g (1.25 mmol) of 4-chlorobenzenesulfonylisocyanate in 25 ml dry toluene the crude product was obtained, wherefrom by chromatography on silica gel column using the solvent system methylene chloride:methanol: 25% ammonia=90:9:1.5, pure 9-deoxo-9-dihydro-9a-N—[N′-(β-cyanoethyl)-N′-(4-chlorobenzenesulfonyl)carbamoyl-γ-aminopropyl]-9a-aza-9a-homoerithromycin A was obtained.

MS(ES⁺)m/z=1051.

### EXAMPLE 18

### 9-Deoxo-9-dihydro-9a-N—[N′-(β-cyanoethyl)-N′-(2-chlorobenzenesulfonyl)carbamoyl-γ-aminopropyl]-9a-aza-9a-homoerithromycin A

From 1.0 g (1.18 mmol) 9-deoxo-9-dihydro-9a-N—[N′-(β-cyanoethyl)-γ-aminopropyl]-9a-aza-9a-homoerithromycin A and 0.27 g (1.25 mmol) of 2-chlorobenzenesulfonylisocyanate in 25 ml dry toluene the crude product was obtained, wherefrom by chromatography on silica gel column using the solvent system methylene chloride:methanol: 25% ammonia=90:9:1.5, pure 9-deoxo-9-dihydro-9a-N—[N′-(β-cyanoethyl)-N′-(2-chlorobenzenesulfonyl)carbamoyl-γ-aminopropyl]-9a-aza-9a-homoerithromycin A was obtained.

MS(ES⁺)m/z=1051.

### EXAMPLE 19

### 9-Deoxo-9-dihydro-9a-N—[N′-(β-cyanoethyl)-N′-(benzenesulfonyl)carbamoyl-γ-aminopropyl]-9a-aza-9a-homoerithromycin A

From 1.0 g (1.18 mmol) 9-deoxo-9-dihydro-9a-N—[N′-(β-cyanoethyl)-γ-aminopropyl]-9a-aza-9a-homoerithromycin A and 0.23 g (1.25 mmol) of benzenesulfonylisocyanate in 25 ml dry toluene the crude product was obtained, wherefrom by chromatography on silica gel column using the solvent system methylene chloride:methanol: 25% ammonia=90: 9:1.5, pure 9-deoxo-9-dihydro-9a-N—[N′-(β-cyanoethyl)-N′-(benzenesulfonyl)carbamoyl-γ-aminopropyl]-9a-aza-9a-homoerithromycin A was obtained.

MS(ES⁺)m/z=1028.

### EXAMPLE 20

### 9-Deoxo-9-dihydro-9a-N—[N′-(β-cyanoethyl)-N′-(4-fluorobenzenesulfonyl)carbamoyl-γ-aminopropyl]-9a-aza-9a-homoerithromycin A

From 1.0 g (1.18 mmol) 9-deoxo-9-dihydro-9a-N—[N′-(β-cyanoethyl)-γ-aminopropyl]-9a-aza-9a-homoerithromycin A and 0.25 g (1.25 mmol) of 4-fluorobenzenesulfonylisocyanate in 25 ml dry toluene the crude product was obtained, wherefrom by chromatography on silica gel column using the solvent system methylene chloride:methanol: 25% ammonia=90:9:1.5, pure 9-deoxo-9-dihydro-9a-N—[N′-(β-cyanoethyl)-N′-(4-fluorobenzenesulfonyl)carbamoyl-γ-aminopropyl]-9a-aza-9a-homoerithromycin A was obtained.

MS(ES⁺)m/z=1014.

### EXAMPLE 21

### 5-O-Desosaminyl-9-deoxo-9-dihydro-9a-N—[N′-(β-cyanoethyl)-γ-aminopropyl]-9a-aza-9a-homoerithronolide A

The solution of 10.0 g (15.7 mmol) 5-O-desosaminyl-9-deoxo-9-dihydro-9a-N-(γ-aminopropyl)-9a-aza-9a-homoerithronolide A and 0.8 ml (15.7 mmol) acrylonitrile in 200 ml methanola was heated at the boiling temperature for a 10 hours and evaporated to dryness and the crude product was obtained wherefrom by chromatography on silica gel column using the solvent system methylene chloride:methanol: 25% ammonia=90:9:1.5 pure 5-O-desosaminyl-9-deoxo-9-dihydro-9a-N—[N′-(β-cyanoethyl)-γ-aminopropyl]-9a-aza-9a-homoerithronolide A was obtained.

MS(ES⁺)m/z=688.

### EXAMPLE 22

### 5-O-Desosaminyl-9-deoxo-9-dihydro-9a-N—[N′-(β-cyanoethyl)-N′-(p-toluenesulfonyl)carbamoyl-γ-aminopropyl]-9a-aza-9a-homoerithronolide A

From 1.0 g (1.46 mmol) 5-O-desosaminyl-9-deoxo-9-dihydro-9a-N—[N′-(β-cyanoethyl)-γ-aminopropyl]-9a-aza-9a-homoerithronolide A and 0.31 g (1.55 mmol) of p-toluenesulfonylisocyanate in 25 ml dry toluene the crude product was obtained, wherefrom by chromatography on silica gel column using the solvent system methylene chloride:methanol: 25% ammonia=90:9:1.5, pure 5-O-desosaminyl-9-deoxo-9-dihydro-9a-N—[N′-(β-cyanoethyl)-N′-(p-toluenesulfonyl)carbamoyl-γ-aminopropyl]-9a-aza-9a-homoerithronolide-de A was obtained.

MS(ES⁺)m/z=883.

### EXAMPLE 23

### 5-O-Desosaminyl-9-deoxo-9-dihydro-9a-N—[N′-(β-cyanoethyl)-N′-(4-chlorobenzenesulfonyl)carbamoyl-γ-aminopropyl]-9a-aza-9a-homoerithronolide A

From 1.0 g (1.46 mmol) 5-O-desosaminyl-9-deoxo-9-dihydro-9a-N—[N′-(β-cyanoethyl)-γ-aminopropyl]-9a-aza-9a-homoerithronolide A and 0.36 g (1.65 mmol) of 4-chlorobenzenesulfonylisocyanate in 25 ml dry toluene the crude product was obtained, wherefrom by chromatography on silica gel column using the solvent system methylene chloride:methanol 25% ammonia=90:9:1.5, pure 5-O-desosaminyl-9-deoxo-9-dihydro-9a-N—[N′-(β-cyanoethyl)-N′-(4-chlorobenzenesulfonyl)carbamoyl-γ-aminopropyl]-9a-aza-9a-homoerithronolide A was obtained.

MS(ES⁺)m/z=889.

### EXAMPLE 24

### 5-O-Desosaminyl-9-deoxo-9-dihydro-9a-N—[N′-(β-cyanoethyl)-N′-(2-chlorobenzene-sulfonyl)carbamoyl-γ-aminopropyl]-9a-aza-9a-homoerithronolide A

From 1.0 g (1.46 mmol) 5-O-desosaminyl-9-deoxo-9-dihydro-9a-N—[N′-(β-cyanoethyl)-γ-aminopropyl]-9a-aza-9a-homoerithronolide A and 0.36 g (1.65 mmol) of 2-chlorobenzenesulfonylisocyanate in 25 ml dry toluene the crude product was obtained, wherefrom by chromatography on silica gel column using the solvent system methylene chloride:methanol: 25% ammonia=90:9:1.5, pure 5-O-desosaminyl-9-deoxo-9-dihydro-9a-N—[N′-(β-cyanoethyl)-N′-(2-chlorobenzenesulfonyl)carbamoyl-γ-aminopropyl]-9a-aza-9a-homoerithronolide A was obtained.

MS(ES⁺)m/z=889.

### EXAMPLE 25

### 5-O-Desosaminyl-9-deoxo-9-dihydro-9a-N—[N′-(β-cyanoethyl)-N′-(o-toluenesulfonyl)carbamoyl-γ-aminopropyl]-9a-aza-9a-homoerithronolide A

From 1.0 g (1.46 mmol) 5-O-desosaminyl-9-deoxo-9-dihydro-9a-N—[N′-(β-cyanoethyl)-γ-aminopropyl]-9a-aza-9a-homoerithronolide A and 0.31 g (1.55 mmol) of o-toluenesulfonylisocyanate in 25 ml dry toluene the crude product was obtained, wherefrom by chromatography on silica gel column using the solvent system methylene chloride:methanol: 25% ammonia=90:9:1.5, pure 5-O-desosaminyl-9-deoxo-9-dihydro-9a-N—[N′-(β-cyanoethyl)-N′-(o-toluenesulfonyl)carbamoyl-γ-aminopropyl]-9a-aza-9a-homoerithronolide A was obtained.

MS(ES⁺)n/z=884.

### EXAMPLE 26

### 5-O-Desosaminyl-9-deoxo-9-dihydro-9a-N—[N′-(β-cyanoethyl)-N′-(benzenesulfonyl)carbamoyl-γ-aminopropyl]-9a-aza-9a-homoerithronolide A

From 1.0 g (1.46 mmol) 5-O-desosaminyl-9-deoxo-9-dihydro-9a-N—[N′-(β-cyanoethyl)-γ-aminopropyl]-9a-aza-9a-homoerithronolide A and 0.36 g (1.65 mmol) of benzenesulfonylisocyanate in 25 ml dry toluene the crude product was obtained, wherefrom by chromatography on silica gel column using the solvent system methylene chloride:methanol: 25% ammonia=90:9:1.5, pure 5-O-desosaninyl-9-deoxo-9-dihydro-9a-N—[N′-(β-cyanoethyl)-N′-(benzenesulfonyl)carbamoyl-γ-aminopropyl]-9a-aza-9a-homoerithronolide A was obtained.

MS(ES⁺)m/z=870.

### EXAMPLE 27

### 5-O-Desosaminyl-9-deoxo-9-dihydro-9a-N—[N′-(β-cyanoethyl)-N′-(4-fluorobenzenesulfonyl)carbamoyl-γaminopropyl]-9a-aza-9a-homoerithronolide A

From 1.0 g (1.46 mmol) 5-O-desosaminyl-9-deoxo-9-dihydro-9a-N—[N′-(β-cyanoethyl)-γ-aminopropyl]-9a-aza-9a-homoerithronolide A and 0.36 g (1.65 mmol) of 4-fluorobenzenesulfonylisocyanate in 25 ml dry toluene the crude product was obtained, wherefrom by chromatography on silica gel column using the solvent system methylene chloride:methanol: 25% ammonia=90:9:1.5, pure 5-O-desosaminyl-9-deoxo-9-dihydro-9a-N—[N′-(β-cyanoethyl)-N′-(4-fluorobenzenesulfonyl)carbamoyl-γ-aminopropyl]-9a-aza-9a-homoerithronolide A was obtained.

MS(ES⁺)m/z=888.

1. Substituted 9a-N—[N′-(benzenesulfonylcarbamoyl)-Y-aminopropyl] and 9a-N-[N′—(P-cyanoethyl)-N′-(benzenesulfonyl)-y-aminopropyl] derivatives of 9-deoxo-9-dihydro-9a-aza-9a-homoerithromycin A and 5-O-desosaminyl-9-deoxo-9-dihydro-9a-aza-9a-homoerithronolide A, novel semisynthetic macrolide antibiotics of the azalide series having antibacterial action of the general formula 1, 1 wherein R represents H or cladinosyl moiety, R1 represents H or (3-cyanoethyl moiety and R2 represents H or fluoro, chloro and methyl group and pharmaceutically acceptable addition salts there of with inorganic or organic acids.

2. Substance according to claim 1, characterized in that R represents cladinosyl group and R1=R2 represent H.

3. Substance according to claim 1, characterized in that R represents cladinosyl group, R1 represents H and R2 represents 4-chloro group.

4. Substance according to claim 1, characterized in that R represents cladinosyl group, R1 represents H and R2 represents 2-chloro group.

5. Substance according to claim 1, characterized in that R represents cladinosyl group, R1 represents H and R2 represents 4-fluoro group.

6. Substance according to claim 1, characterized in that R represents cladinosyl group, R1 represents H and R2 represents 4-methyl group.

7. Substance according to claim 1, characterized in that R represents cladinosyl group, R1 represents H and R2 represents 2-methyl group.

8. Substance according to claim 1, characterized in that R═R′═R2 represent H.

9. Substance according to claim 1, characterized in that R=Ri represent H and R2 represents 4-chloro group.

10. Substance according to claim 1, characterized in that R=Ri represent H and R2 represents 2-chloro group.

11. Substance according to claim 1, characterized in that R=R1 represent H, and R2 represents 4-fluoro group.

12. Substance according to claim 1, characterized in that R═R1 represent H, and R2 represents 4-methyl group.

13. Substance according to claim 1, characterized in that R═R′represent H, and R2 represent 2-methyl group.

14. Substance according to claim 1, characterized in that R represents cladinosyl group, Ri represents (3-cyanoethyl group and R represents H.

15. Substance according to claim 1, characterized in that R represents cladinosyl group, R1 represents p-cyanoethyl group, and R2 represents 4-chloro group.

16. Substance according to claim 1, characterized in that R represents cladinosyl group, R1 represents p-cyanoethyl group, and R2 represents 2-chloro group.

17. Substance according to claim 1, characterized in that represents cladinosyl group, R1 represents p-cyanoethyl group, and R2 represents 4-fluoro group.

18. Substance according to claim 1, characterized in that R represents cladinosyl group, R1 represents p-cyanoethyl group, and R2 represents 4-methyl group.

19. Substance according to claim 1, characterized in that R represents cladinosyl group, Ri represents (3-cyanoethyl group, and R2 represents 2-methyl group.

20. Substance according to claim 1, characterized in that R represents H, and. R1 represents p-cyanoethyl group.

21. Substance according to claim 1, characterized in that R represents H, Ri represents—cyanoethyl group, and R2 represents 4-chloro group.

22. Substance according to claim 1, characterized in that R represents H, Ri represents—cyanoethyl group, and R represents 2-chloro group.

23. Substance according to claim 1, characterized in that R represents H, Ri represents—cyanoethyl group, and R2 represents 4-fluoro group.

24. Substance according to claim 1, characterized in that R represents H, R represents—cyanoethyl group, and R2 represents 4-methyl group.

25. Substance according to claim 1, characterized in that R represents H, R1 represents—cyanoethyl group, and R2 represents 2-methyl group.

26. Process for the preparation of 9a-N—[N′-(benzenesulfonyl)carbamoyl-y-aminopropyl] and 9a-N—[N′—(P-cyanoethyl)-N′-(benzenesulfonyl)carbamoyl-y-aminopropyl] derivatives of 9-deoxo-9-dihydro-9a-aza-9a-homoerithromycin A and 5-O-desosaminyl-9-deoxo-9-dihydro-9a-aza-9a-homoerithronolide A of the general formula 1, 1 wherein R represents H or cladinosyl group, R1 represents H or p-cyanoethyl group, and R represents H or fluoro, chloro and methyl group, characterized in that 9a-N-(y-aminopropyl) and 9a-N—[N′—(P-cyanoethyl)-y-aminopropyl] derivatives of 9-deoxo-9-dihydro-9a-aza-9a-homoerithromycin A and 5-O-desosaminyl-9-deoxo-9-dihydro-9a-aza-9a-homoerithronolide A general formula 2, wherein R represents H and cladinosyl group and R1 represents H and p-cyanoethyl group is reacted with substituted phenylsulfonylisocyanate general formula 3 3 wherein R2 represents H, chloro, fluoro and methyl group, in toluene, xylene or some other aprotic solvents, at a temperature 0-110° C. and then, if appropriate, to a reaction with inorganic or organic acids.

27. Pharmaceutical composition comprising a pharmaceutically acceptable carrier and an antibacterially effective amount of the substances according to claim 1.

28. Use of a substance according to claim 1 for preparing compositions for sterilization rooms and medical instruments as well as for protection of wall and wooden coatings.
